(19) United States
(12) Patent Application Publication
(10) Pub. No.: US 2004/0247696 A1
(43) Pub. Date: Dec. 9, 2004

(54) Methods of using electron active compounds for managing conditions afflicting mammals

(76) Inventor: Marvin S. Antelman, Rehovoth (IL)

Correspondence Address: BANNER & WITCOFF, LTD., 28 STATE STREET, 28th FLOOR, BOSTON, MA, 02109-9601, US

(21) Appl. No.: 10/482,222

(22) PCT Filed: Jul. 3, 2002
(86) PCT No.: PCT/US02/21232

Related U.S. Application Data
(60) Provisional application No. 60/302,656, filed on Jul. 5, 2001.

(51) Int. Cl. A61K 33/24; A61K 33/38; A61K 33/34

(52) U.S. Cl. 424/617; 424/618; 424/635; 424/653

(57) ABSTRACT

The present invention relates to a method of preventing, treating, or managing a condition of an animal, such as a mammal. The animal is administered with a therapeutically effective amount of at least one electron active compound, or a pharmaceutically acceptable derivative thereof, that has at least two polyvalent cations, at least one of which has a first valence state and at least one of which has a second different valence state, to prevent, treat, or manage the condition, or a symptom thereof.

A multivalent metal oxide, such as Ag(I,III), Cu(I,III), Pr(III,IV), and Bi(III,V) oxides or a pharmaceutically acceptable derivative thereof, may be administered to the animal in an amount and for a period of time which is therapeutically effective to prevent, treat, and/or manage such a condition(s) afflicting the animal.

### CROSS-REFERENCE TO RELATED APPLICATIONS

This application claims priority to the U.S. Provisional Patent No. 60/302,656, filed Jul. 5, 2001 and to International Publication No. WO 03/003809 A2, filed Jul. 3, 2002, and published Jan. 16, 2003, the entire contents of each of which are hereby incorporated by reference hereto.

### FIELD OF THE INVENTION

The invention relates to pharmaceutical compositions and methods of using such compositions, for the prevention, treatment, and management of conditions or diseases afflicting animals, such as mammals.

### BACKGROUND OF THE INVENTION

A number of diseases impact the health of mammals, such as livestock and pets. Foot and mouth disease (FMD) is an acute, highly contagious infection of cloven hooved animals by viruses of the family Picornaviridae, genus Aphthovirus. Hosts include members of the genus bovidae, such as cattle, zebus, domestic buffaloes, yaks, sheep, goats, swine, all wild ruminants and suidae. Camelidae, the camels, dromedaries, llamas and vicunas have lower susceptibility. People can be infected through skin wounds or the oral mucosa by handling diseased stock, the virus in the laboratory, or by drinking infected milk but not by eating meat from infected animals. The human infection is generally temporary and mild. The disease is endemic in parts of Asia, Africa, the Middle East and South America

There are at least seven immunologically distinct serotypes: A, O, C, SAT1, SAT2, SAT3 and Asia 1. The virus (FMDV) is sensitive to environmental influences, being inactivated by pH less than 6.0 or greater than 9.0, sunlight and dessication, but can survive for long periods at freezing temperatures. The virus survives in lymph nodes and bone marrow at neutral pH, but is destroyed in muscle when pH is less than 6.0 (i.e., after rigor mortis). The virus can persist in contaminated fodder and the environment for up to 1 month, depending on temperature and pH conditions.

FMDV is resistant to many of the most widely used biocides, including iodophores, quaternary ammonium compounds, hypoclorite and phenol, especially in the presence of organic matter. Thus, this highly contagious disease is difficult to contain and may spread over great distances with movement of infected or contaminated animals, products, objects and people. Pigs, for example, are mainly infected ingesting infected food. Waste feeding has been associated with outbreaks. Cattle are mainly infected by inhalation, often from pigs, which excrete large amounts of virus by respiratory aerosols and are considered highly important in disease spread. Large amounts of virus are excreted by infected animals before clinical signs are evident, and winds may spread the virus over long distances.

The incubation period is 2 to 21 days (average 3 to 8) although virus is shed before clinical signs develop. Rate of infection (morbidity) can reach 100%, however mortality can range from 5% (adults) to 75% (suckling pigs and sheep). Recovered cattle may be carriers for 18 to 24 months; sheep for 1 or 2 months. Pigs are not carriers.

Clinical signs in cattle are salivation, depression, anorexia and lameness caused by the presence of painful vesicles (blisters) in the skin of the lips, tongue, gums, nostrils, coronary bands, interdigital spaces and teats. Fever and decreased milk production usually precede the appearance of vesicles. The vesicles rupture, leaving large denuded areas which may become secondarily infected. In pigs, sheep and goats the clinical signs are similar but milder. Lameness is the predominant sign.

Because of the range of species affected, the high rate of infectivity, and the fact that virus is shed before clinical signs occur, FMD is one of the most feared reportable disease in North America. An outbreak of FMD would, (and in the past) cost millions of dollars in lost production, loss of export markets, and loss of animals during eradication of the disease.

Current attempts at prophylaxis do not include effective means of treatment.

Feline Leukemia Virus (FeLV) is a viral disease of cats and the leading killer of cats, claiming nearly 1 million pets per year. A retrovirus, FeLV may attack in two ways: (i) attacks tissues and organs of immune system which, when weakened, leaves animal susceptible to numerous infectious disease, such as anemia, eye problems, respiratory disease, skin problems, oral infections and Feline Infectious Peritonitis—typically chronic and ultimately fatal; or (ii) causes infection that leads to development of leukemia.

Virus circulates in blood, either free in the serum or within infected white blood cells. About 30% of infected cats become persistently infected when virus infects bone marrow, foreclosing effective treatment and dramatically shortening life (⅓ of infected cats will die within 6 months, and most within 4 years). The longer living ones are potential carriers. Prolonged cat-to-cat contact is required for efficient spread, because the virus is rapidly inactivated by warmth and drying.

The virus is shed through saliva, urine and feces of infected cat, and can be spread by licking or biting. Pregnant cats can spread virus to kittens in womb or by nursing and grooming them. Healthy cats can contact virus by sharing food dishes or litter boxes with infected cat. Cats that go outside or are in contact with infected cats are at greatest risk. Common clinical signs include anemia, jaundice, depression, weight loss, decreased appetite, diarrhea or constipation, blood in the stool, enlarged lymph nodes, respiratory distress, decreased stamina, excessive drinking and urination, fetal resorption, abortion, infertility, birth of “fading” kittens, and a syndrome resembling panleukopenia (“cat distemper”). Cancer occurs in some infected cats. The illness is diagnosed through simple blood test. No evidence exists that FeLV is carried by, or causes illness in, dogs or humans.

Uninfected cats can be vaccinated by several available vaccines that either contain inactivated whole virus or subunit protein of the virus. Vaccines are reasonably effective in preventing FeLV but not 100%.

There is no cure for FeLV infection or disease. A variety of chemotherapeutic regimens have been developed, and in certain cases those regimens can produce a temporary remission of from several weeks to several months. Various antiviral compounds including interferon may also be used, which are generally safer than chemotherapeutic agents and may reduce amount of virus in blood and extend period of remission. Steroid therapy (such as with prednisolone) acts to decrease number of some circulating white blood cells (lymphocytes), including cancerous lymphocytes, and may also act directly against cells of some solid tumors (such as lymphosarcoma) caused by FeLV. Steroids also inhibit cells that normally destroy senescent red blood cells, helping to combat anemia and excessive red blood cell destruction that often accompany FeLV. However, both FeLV and steroids suppress the immune system, and cats undergoing steroid therapy are especially vulnerable to other infections.

Respiratory disease affecting feedlot cattle causes tremendous losses yearly to the cattle industry. Calves are the most severely affected, and a large number of these calves die. This disease is associated with pathogenic microorganisms, particularly Pasteurella species, and various stresses, such as transportation and overcrowding. Shipping fever is the most economically important respiratory disease associated with Pasteurella species.

The disease is characterized by sudden onset, usually within two weeks of stress. The symptoms include dyspnea, cough, ocular and nasal discharge, inappetance and rapid weight loss, fever, increased lung sounds, immunosuppression, general depression, viral and/or bacterial infection of the lungs. Various bacteria and viruses have been isolated from affected animals including Pasteurella spp., bovine herpes virus 1, parainfluenza-3 virus, bovine respiratory syncytial virus and Mycoplasma species.

The disease typically affects 15-30% of exposed animals and the resulting deaths are typically 2-5% of the exposed population. Exposure of the animal to stress, plus infection with a variety of viruses, as described above, appears to make the animal susceptible to fibrinous pneumonia caused by P. haemolytica, and to a lesser extent, Pasteurella multocida. For a general background on shipping fever see Yates, W. D. G. (1982) Can. J. Comp. Med. 46:225-263. P. haemolytica also causes enzootic pneumonia and can infect a wide range of animals, in addition to cattle, including economically important species such as sheep, swine, horses and fowl. P. haemolytica is also frequently found in the upper respiratory tract of healthy animals. Pneumonia develops when the bacteria infects the lungs of these animals. Protection against Pasteurella-associated diseases is therefore economically important to the agricultural industry.

Feline immunodeficiency virus (FIV) is a retrovirus (of same family as FeLV and HIV) that attacks immune system of cats, leading to inability to fight off infections and cancers. Retroviruses are species-specific, so FIV does not infect humans. Retroviruses are comprised of RNA that, in the host, is transcribed into DNA and incorporated into the DNA of the host's cells. FIV is fragile, being easily inactivated by ultraviolet light, heat, detergents and drying.

FIV is not transmitted by prolonged closed contact (as in FeLV), but is shed in saliva and transmitted by bite wounds. Transmission in utero or through milk is rare, and usually only occurs if the mother is initially infected during gestation or lactation. FIV is found worldwide in domestic cats, and also infects wild felines including snow leopards, lion, tigers, jaguars, Florida panthers and bobcats. In the U.S., about 1 to 8% of apparently healthy cats are infected, with males being twice are likely to become infected as females.

Infection is in 3 stages: (1) initial or acute state often characterized by fever, swollen lymph nodes and susceptibility to skin or intestinal infections (generally occurs 4 to 6 weeks after exposure to virus; (2) latent or subclinical stage in which see no signs of disease (this stage can last for many years) but immune system may slowly be destroyed; and (3) third, final, AIDS-like stage (occurs most commonly at 5 to 12 years of age and typically lasts up to a year ending in death) during which immune system is not functioning properly and cat is very prone to infection which are usually chronic and opportunistic, and may be bacterial, fungal or parasitic such as upper respiratory tract infections with sneezing and nasal discharge (about 30% of infected cats), intestinal infections (e.g., diarrhea), and skin/ear diseases (caused by parasites, yeast and bacteria). Certain cancers may develop (lymphoma and leukemia are five times (5×) more likely than in uninfected cats); also can have neurological disease (e.g., change in behavior, loss of house-training, dementia) or anemia.

There are many antiviral medications for people with HIV infection, but currently none are routinely and effectively used in FIV-infected cats. Some drugs that directly affect the immune system have been used with variable but encouraging results, including Propionibacterium acnes (ImmunoRegulin), low doses of oral human alpha interferon and an aloe derivative called Acemannan. Cats with FIV-related disease are treated according to the sign of disease they are showing. Testing and segregating infected cats is the only means by which FIV infection can be controlled.

Metal oxides, such as electron active metal oxides comprising multivalent silver cations, have been disclosed for various uses, as they are reported to be non-toxic to animals and humans. M. Antelman, “Anti-Pathogenic Multivalent Silver Molecular Semiconductors,” Precious Metals, vol. 16:141-149 (1992); M. Antelman, “Multivalent Silver Bactericides,” Precious Metals, vol. 16:151-163 (1992). For example, tetrasilver tetroxide activated with an oxidizing agent is disclosed for use in bactericidal, fungicidal, and algicidal use, such as in municipal and industrial water treatment applications and for the treatment of AIDS.

Thus, it is desired to find pharmaceutical compositions and methods for preventing, treating, or managing one or more conditions associated with animals.

### SUMMARY OF THE INVENTION

The present invention relates to a method for preventing, treating, or managing one or more diseases in an animal, which is preferably a mammal. Preferably, the animal is a domesticated mammal, such as livestock, cattle, or dairy producing cattle. In one embodiment, the mammal is a ruminant of the genus bos, such as a cow, a ruminant of the genus Ovis, such as a sheep, a ruminant of the genus Capra, such as a goat. In another embodiment, the mammal is a member of the family felidae, such as a cat. The mammal can be a member of the family Suidae, such as a pig. The mammal can be a member of the genus Equus, such as a horse. Members of the family canidae, such as dogs, can also be treated according to the invention.

The method preferably comprises administering at least one metal oxide compound or a pharmaceutically acceptable derivative thereof, to the animal. The metal oxide compound or derivative thereof preferably comprises a first metal cation having a first valence state and a second metal cation having a second, different valence state, such as, for example, an electron active metal oxide compound. The at least one metal oxide compound or a pharmaceutically acceptable derivative thereof is preferably administered in an amount and for a period of time which is therapeutically effective to treat such condition(s).

In a preferred embodiment, the at least one metal oxide compound or pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide. In one embodiment of the invention, the metal oxide compound comprises essentially no Ag(I,III) oxide. In another embodiment of the invention, the metal oxide compound comprises no Ag(I,III) oxide.

The metal oxide compound or derivative thereof is preferably substantially free of added persulfate.

The invention is preferably adapted to preventing, treating, or managing systemic conditions. Conditions suitable for treatment include Actinobacillosis, Anaplasmosis, Bovine babesiosis, Bovine ephemeral fever (BEF), Bovine brucellosis, Boophilus microplus, Haemorrhagic septicaemia (HS), Contagious bovine pleuropneumonia (CBPP), Rinderpest, Bovine tuberculosis (bovine TB), calf diphtheria, foot-and-mouth disease, bovine respiratory disease, feline immunodeficiency virus, feline leukemia, and cancer.

The metal oxide compound is preferably administered via intravenous injection or infusion. The intravenous injection or infusion is preferably subcutaneous, intramuscular, or comprises infusion into the bloodstream of the animal. Preferably, the administration provides an amount of the metal oxide sufficient to provide about 1 to about 1000 ppm of the metal oxide compound or derivative thereof in the bloodstream. The metal oxide is preferably administered via infusion over a period of time sufficient to inhibit adverse side effects, such as over a time period of from about 30 minutes to about 300 minutes.

The metal oxide compound or derivative thereof may preferably be administered by a controlled release vehicle. The controlled release vehicle is preferably implanted in the body at a location suitable for providing a therapeutically effective amount of metal oxide compound or derivative thereof to the animal, preferably, without affecting proper functioning of the animal's liver.

The metal oxide compound or derivative thereof preferably comprises a first metal cation having a first valence state and a second metal cation having a second, different valence state. In a preferred embodiment, the at least one metal oxide compound or pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

The metal oxide compound or derivative thereof is preferably essentially free of added persulfate.

The method of the invention is preferably suitable for preventing, treating, or managing conditions, such as hoof and mouth disease, feline leukemia, respiratory illness, such as those associated with infection by Pasteurella species of bacteria, and immunodeficiency diseases, such as feline immunodeficiency virus.

In another embodiment, the administering preferably comprises a carrier medium in which the at least one metal oxide compound or pharmaceutically acceptable derivative thereof, is dispersed. Preferably the therapeutically effective amount of the metal oxide or derivative thereof is from about 50 ppm to 500,000 ppm, such as from about 400 ppm to about 100,000 ppm, based on the weight of the carrier medium. The carrier medium may preferably comprise petroleum jelly. The administering of the composition is preferably topical or transdermal, such as directly to the skin.

Preferably, the at least one metal oxide compound or pharmaceutically acceptable derivative thereof, further comprises a thixotropic agent sufficient to increase adherence of the composition to the skin without excessive runoff.

The at least one metal oxide compound or pharmaceutically acceptable derivative thereof may, preferably, be administered in the form of a powder, such as in the form of metal oxide crystals. The administering of the powder is preferably topical or transdermal, such as directly to the skin. Preferably the metal oxide or derivative thereof is administered at a dosage level of about 10 mg to 500 mg per cm2 of skin surface. A preferred embodiment of a composition suitable for application as a powder comprises about 5% metal oxide, such as tetrasilver tetroxide, and about 95% bismuth subgallate.

An embodiment of the invention relates to a method of preventing, treating, or managing a condition of a mammal, which method comprises administering a therapeutically effective amount of at least one electron active compound, or a pharmaceutically acceptable derivative thereof, that has at least two polyvalent cations, at least one of which has a first valence state and at least one of which has a second different valence state, to prevent, treat, or manage the condition, or a symptom thereof.

Definitions Section

Suitable definitions are provided herein for some of the terms relating to the present invention.

The terms “patient” or “subject” as used herein refer to animals, particularly to mammals.

As used herein, the terms “adverse effects,” “adverse side effects,” and “side effects” include, but are not limited to, staining of the skin, headache, dry mouth, constipation, diarrhea, gastrointestinal disorders, dry skin, staining of the skin, hepatomegaly, fever, fatigue, weight loss and the like.

The phrase “therapeutically effective amount” when used herein in connection with the compositions and methods of the invention, means that amount of metal oxide composition, or a derivative thereof, which, alone or in combination with other drugs or treatment modalities, provides a therapeutic benefit in the prevention, treatment, or management, of a condition, such as a pathogen induced disease, or a symptom or related condition thereof. Preferably, the therapeutically effective amount of a component yields the desired therapeutic benefit without undue adverse side effects (such as toxicity, irritation, or allergic response) commensurate with a reasonable benefit/risk ratio when used in the manner of this invention.

The terms essentially free and “substantially free” mean less than about 10 weight percent, preferably less than about 5 weight percent, more preferably less than about 1 weight percent, and most preferably less than about 0.1 weight percent of added persulfate is present according to the invention. In another embodiment, the terms refer to the same amounts of other added oxidizing agents present in the compositions.

The term “controlled-release component” in the context of the present invention is defined herein as a compound or compounds, including polymers, polymer matrices, gels, permeable membranes, liposomes, microspheres, or the like, or a combination thereof, that facilitates the controlled-release of the active ingredient (e.g., tetrasilver tetroxide) in the pharmaceutical composition.

The term “about,” as used herein, should generally be understood to refer to both numbers in a range of numerals. Moreover, all numerical ranges herein should be understood to include each whole integer within the range.

### DETAILED DESCRIPTION OF THE PREFERRED EMBODIMENTS

It has now been discovered that pharmaceutical compositions comprising at least one oxide compound or a pharmaceutically acceptable derivative thereof can be used as advantageous active ingredients in the prevention, treatment, or management of various systemic conditions, such as those caused by infection with pathogens, such as one or more viruses, parasites, and or bacteria. The oxide compound preferably comprises a metal oxide, such as an electron active metal oxide. The metal oxide compound or pharmaceutically acceptable derivative thereof preferably comprise a first metal cation having a first valence state and a second metal cation having a second, different valence state. One of ordinary skill in the art understands that, in general, the valence state of a species, such as a metal cation, is related to the charge associated with or assigned to the species.

Conditions affecting mammals, such as cattle and domesticated animals, are particularly suited for treatment according to the invention. Among diseases that can be treated with the composition of the invention are Actinobacillosis, Anaplasmosis, Bovine babesiosis, Bovine ephemeral fever (BEF), Bovine brucellosis, Boophilus microplus, Haemorrhagic septicaemia (HS), Contagious bovine pleuropneumonia (CBPP), Rinderpest, Bovine tuberculosis (bovine TB), calf diphtheria, foot-and-mouth disease, bovine respiratory disease, feline immunodeficiency virus, feline leukemia, and cancer.

Actinobacillosis, is a disease characterised by the presence of granulomas with pus containing small, hard yellow to white granules. The disease is caused by infection with Actinobacillus lignieresi. A similar condition can be produced by infection with Actinomyces bovis (‘actinomycosis’). However, actinobacillosis tends to affect soft tissue while actinomycosis tends to affect bone.

Anaplasmosis, which is a form of ‘tick fever’ in cattle, is caused by the rickettsia Anaplasma marginale. It is characterised by initial high fever and progressive anaemia.

Bovine babesiosis is a major tick-borne disease of cattle due to protozoan parasites (Babesia sp.). In tropical areas the agents are Babesia bovis and Babesia bigemina.

Bovine ephemeral fever (BEF) is an arboviral disease of cattle, characterised by an acute fever of short duration, with high morbidity and low mortality.

Bovine brucellosis is a highly contagious bacterial disease causing late term-abortion and infertility in cattle. The disease is also a serious zoonosis, causing undulant fever in humans.

The cattle tick Boophilus microplus is a significant ectoparasite of cattle and a vector for important diseases such as babesiosis and anaplasmosis.

Contagious bovine pleuropneumonia (CBPP) is an acute, subacute or chronic respiratory disease of cattle caused by a Mycoplasma.

Haemorrhagic septicaemia (HS) is a specific form of acute pasteurellosis of cattle and buffaloes with a high mortality rate in infected animals. It is regarded as one of the most serious diseases of large ruminants in south east Asia.

Rinderpest, which results from a viral infection, is an acute, highly contagious disease of cattle. It is characterised by inflammation and necrosis of mucous membranes and a very high mortality rate.

Bovine tuberculosis (bovine TB) is a contagious chronic disease of cattle associated with progressive emaciation and tubercle (granuloma) formation involving the respiratory or alimentary system. As well as being of great economic importance to the livestock industry, because humans can be infected, it is also an important public health issue. Cattle and buffaloes are the principal hosts for Mycobacterium bovis although pigs can also become infected.

Calf diphtheria, which is caused by infection with Spherophorus necrophorus, a soil organism, produces symptoms include breathing difficulty, drooling, problems with eating and drinking, and dead tissue.

Foot and mouth disease is caused by small filtrable viruses, symptoms include water blisters in the mouth or on the skin by the claws on the hoof and on the teats and udders, also fever.

Pathogens which may be killed by, or the growth or proliferation of which may be halted, diminished, or inhibited by, the electron active metal oxides of the present invention include, but are not limited to, gram-positive bacilli and cocci; gram-negative bacilli and cocci; acid-fast bacteria; other bacteria; fungi; parasitic microbes, e.g., protozoa; and viruses.

Examples of gram-positive bacilli and cocci include, but are not limited to, Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae, and combinations thereof.

Examples of gram-negative bacilli and cocci include, but are not limited to, Afipia felis, Bacteriodes, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis, and combinations thereof.

Examples of acid-fast bacteria include, but are not limited to, Myobacterium avium, Myobacterium leprae, Myobacterium tuberculosis, and combinations thereof.

Examples of other bacteria not falling into the other three categories include, but are not limited to, Bartonella henseiae, Chlamydia psittaci, Chlamydia trachomatis, Coxiella bumetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci, and combinations thereof.

Examples of fungi include, but are not limited to, Aspergilli, Candidae, Candida albicans, Coccidioides immitis, Cryptococci, and combinations thereof.

Examples of parasitic microbes include, but are not limited to, Balantidium coli, Cryptosporidium parvum, Cyclospora cayatanensis, Encephalitozoa, Entamoeba histolytica, Enterocytozoon bieneusi, Giardia lamblia, Leishmaniae, Plasmodii, Toxoplasma gondii, Trypanosomae, trapezoidal amoeba, and combinations thereof.

Examples of viruses include, but are not limited to, Arboviruses, Ebola virus, Guanarito virus, Hanta virus, Hantaan virus, Hepatitis A, Hepatitis B, Hepatitis C, Hepatitis E, other Hepatitis viruses, Herpes-type viruses, Poliovirus, West Nile virus, Echo virus, and combinations thereof.

Preferably, the at least one metal oxide compound or a pharmaceutically acceptable derivative comprises at least one electron active metal oxide compound, such as, for example, at least one of Bi(III,V) oxide, Co(II,III) oxide, Cu(I,III) oxide, Fe(II,III) oxide, Mn(II,III) oxide, Pr(III,IV) oxide, or Ag(I,III) oxide. Preferred compounds of the invention comprise at least one metal tetroxide, such as silver tetroxide. The terms metal tetroxide and metal tetraoxide, are synonymous as used herein.

In one preferred embodiment, the metal oxide compound compositions are substantially free of added persulfate or other added oxidizing agents, since, when applied topically, such agents may cause adverse effects, such as skin irritation and skin over-drying. In another preferred embodiment, the compositions are substantially free of any oxidizing agents.

In one embodiment, the compositions include a molecular scale device comprising at least one crystal of a metal oxide compound. A plurality of these metal oxide crystals, such as on the order of trillions, may be employed in various pharmaceutical formulations and therapies to effectuate the prevention, treatment, and/or management of various conditions. The compositions of the invention include powders comprising metal oxide crystals of the invention.

The compositions and methods of the invention advantageously provide a desired effect such as preventing, treating, or managing a systemic condition. “Management,” as used herein, includes controlling one or more conditions that cannot be cured completely, reducing the severity of affliction of such conditions, and the like.

Preferred metal oxides of the invention comprise a first metal cation having a first valency state and a second metal cation having a second valency state, which differs from the first valency, preferably by at least one charge. The first and second metal cations are preferably the same metal. Without being bound by theory, it is believed that the metal oxides of the present invention operate by transferring electrons between cations of differing valency, the electrons contributing to the death of the pathogens by traversing the cell membrane. By way of non-limiting example, it is believed that the crystal lattice of a silver tetroxide (Ag4O4) molecular device operates against pathogens by transferring electrons from its two monovalent silver ions to the two trivalent silver ions in the crystal, contributing to the death of the pathogens by traversing their cell membrane surface. This in effect “electrocutes” the cells. The electrons are forced out of their balanced crystals by such labile groups as NH, NH2, S—S, and SH associated with the cellular surface. Cells of the host organism (ie mammal) are not believed to be affected, because they are not believed to proliferate fast enough to expose these labile bonds.

The metal oxides of the invention are preferably stable as determined by the dissociation constants of the compounds. For example, the dissociation constant (KA) of Ag4O4 is 7.9×10-13. Therefore the molecule is not believed to be disturbed unless more stable complexes are formed with such ligands as those associated with the cancer cell membrane surface in a dynamic state. Indeed, the end result of the electron transfer, which is a redox reaction, is believed to result in the metal ions of a lower valency being oxidized to a higher valency state and metal ions of a higher valency state being reduced to a lower valency state.

Returning to the non-limiting example of silver tetroxide, it is believed that monovalent Ag ions are oxidized to Ag(II) and the trivalent Ag ions are reduced to the same end product, Ag(II). Accordingly, the well-known affinity of monovalent silver for certain elements such as sulfur and nitrogen is believed to be far exceeded here, for divalent silver is believed to not merely bind to these elements as does silver, but to actually form chelate complexes with their ligands. The molecular crystal attraction for the cell membrane surfaces is thus believed to be driven by powerful covalent bonding forces.

The electron transfer occurring in the example of silver tetroxide can be depicted by the following redox half reactions:

Ag+-e=Ag+2

Ag+3+e=Ag+2

It was found by rigorous testing that certain silver tetroxide containing-compositions were comparatively non-toxic in comparison to monovalent silver salts. Prior to the acceptance of the oxide in commerce, for which EPA registration No. 3432-64 was obtained, it was necessary for the oxide to undergo a series of toxicity tests. A 3% concentrate was used and evaluated by a certified laboratory employing good laboratory practice (GLP) according to the Code of Federal Regulations for this purpose.

The results were as follows:

| | |
|---|---|
| Acute Oral Toxicity | LD50 Greater than 5,000 mg/Kg |
| Acute Dermal Toxicity | LD50 Greater than 2,000 mg/Kg |
| Primary Eye Irritation | Mildly irritating |
| Primary Skin Irritation | No irritation |
| Skin Sensitization | Non-Sensitizing |

Subsequent evaluations conducted according to the invention showed that unless an animal was prone to silver allergies, the pure tetroxide compositions according to the invention could be applied to, for example, the skin without any ill effects or evidence of irritation, despite the fact that the compositions of the invention can be a powerful oxidizing agent. This can perhaps be explained by the stability manifested by the above-noted KA of the silver compositions. Accordingly, in a preferred embodiment, the metal oxides of the invention are applied directly in a powder or composition form to afflicted areas, such as the skin, cervix, or cervical pelvic region of an afflicted animal. Preferred routes of administration include topically and application to mucosa. Application can be made, for example, digitally or using a suitable applicator.

One embodiment of the present invention relates to compositions and methods of using the metal oxide compositions of the invention while minimizing the amount of additional oxidizer, such as persulfate. It has been found in accordance with the present invention that the additional oxide is not required and in some circumstances is undesirable when the oxide is applied to, for example, the skin or cervix, in part due to the undesirable side effect of irritation. In one embodiment, the compositions are substantially free of added persulfates, while in a preferred embodiment, the compositions are completely free of added persulfates. In one preferred embodiment, the compositions are substantially free of added oxidizer, while in another preferred embodiment they are completely free of added oxidizer. The aforementioned compositions may be applied topically or to mucosa associated with, for example, the skin, cervix, vagina, anus, colon, or other mucosa.

The metal oxide compound, such as tetrasilver tetroxide, may be black in color, such that care must be taken when formulating suitable topical pharmaceutical compositions according to the invention to inhibit or avoid blackening or staining of the skin. Without being bound by theory, it is believed that larger amounts of the silver tetroxide composition promote increased staining. Thus, in one embodiment, the pharmaceutical compositions preferably have an insufficient amount of metal oxide compound to cause visible skin staining.

Where the metal oxide compositions according to the invention are applied to the skin, they may be combined with a carrier at an amount from about 5 ppm to 500,000 ppm, more preferably from about 50 ppm to 250,000 ppm of the metal oxide composition, based on the weight of the carrier. In various embodiments, the compositions are provided in amounts from about 400 ppm to 100,000 ppm, from about 1,000 ppm to 70,000 ppm, from about 10,000 ppm to 50,000 ppm, or from about 20,000 ppm to 40,000 ppm. In one preferred embodiment, the compositions are formulated with about 25,000 ppm to 35,000 ppm of metal oxide. It will be readily understood by those of ordinary skill in the art that the administration of about 0.05 to about 0.25 g of metal oxide to an adult animal such as a cow weighing about 1800 pounds provides about 1 ppm of the metal oxide in the bloodstream of the cow. In another embodiment, the concentration of the metal oxide crystals dispersed in the carrier ranges from about 0.1 to 10% by weight, more preferably from about 0.25 to 5% by weight and most preferably from about 2 to 4% by weight. The compositions, when applied topically, can be applied to the skin about 1 to 3 times per day until the condition is suitably cured or satisfactorily controlled. In one embodiment, the composition may generally be topically applied at a dosage level of from about 1 mg to 1000 mg per cm2 of skin surface, preferably about 10 mg to 500 mg per cm2 of skin surface.

A preferred carrier for topical formulations and administration includes petroleum jelly, such as white petroleum jelly. For example, a suitable white petroleum jelly is available from Penreco of Houston, Tex.

A preferred mode of application of the oxide of the invention is as an ointment. Suitable formulations include, but are not limited to, salves and the like. If desired, these may be sterilized or mixed with auxiliary agents, e.g., thixotropes, stabilizers, wetting agents, and the like. Preferred vehicles include ointment bases, e.g., polyethylene glycol-1000 (PEG-1000); conventional ophthalmic vehicles; creams; and gels, as well as petroleum jelly and the like.

Leukemia, which is a malignant overproduction of white blood cells, lymphoma, and metastasized melanoma which has proliferated from skin via blood and/or the lymphatic system can also be treated.

Different therapeutically effective amounts and delivery systems may be applicable for each disorder, as will be readily known or determined by those of ordinary skill in the art. Suitable delivery systems for delivering compositions of the invention to cattle are disclosed in U.S. Pat. No. 6,074,657, which is hereby incorporated herein in its entirety.

A preferred metal oxide for use according to the invention, tetrasilver tetroxide, has been commercially sold under the poorly named “Ag(II) OXIDE” tradename. It may also be obtained from Aldrich Chemical Co., Milwaukee, Wis. The chemical synthesis of silver oxide compounds according to the invention can be performed according to the method described on page 148 in M. Antelman, “Anti-Pathogenic Multivalent Silver Molecular Semiconductors,” Precious Metals, vol. 16:141-149 (1992) by reacting silver nitrate with potassium peroxydisulfate according to the following equation in alkali solutions:

4 AgNO₃+2 K₂S₂O₈+8 NaOH═══>Ag₄O₄+3 Na₂SO₄+K₂SO₄+2 NaNO₃+2 KNO₃+4H₂O

The magnitude of a prophylactic or therapeutic dose of metal oxide composition(s), or a derivative thereof, in the acute or chronic management of diseases and disorders described herein will vary with the severity of the condition to be prevented, treated, or managed and the route of administration. For example, oral, mucosal (including vaginal and rectal), parenteral (including subcutaneous, intramuscular, bolus injection, and intravenous, such as by infusion), sublingual, transdermal, nasal, buccal, and like may be employed. Dosage forms include tablets, troches, lozenges, dispersions, suspensions, suppositories, solutions, capsules, soft elastic gelatin capsules, patches, and the like. The dose, and perhaps the dose frequency, will also vary according to the age, body weight, and response of the animal being treated. Suitable dosing regimens can be readily selected by those of ordinary skill in the art with due consideration of such factors.

In general, for topical and mucosal application, such as application to the skin or cervix, the total daily dosage for the conditions described herein can be from about 1 mg to 5000 mg of the metal oxide or derivative thereof, while in another embodiment, the daily dosage can be from about 2 mg to 4000 mg of the metal oxide composition. A unit dosage can include, for example, 30 mg, 60 mg, 90 mg, 120 mg, or 200 mg of metal oxide composition. Preferably, the active ingredient is administered in single or divided doses from one to four times a day.

In another embodiment, the compositions are administered by an oral route of administration. The oral dosage forms may be conveniently presented in unit dosage forms and prepared by any methods available to those of ordinary skill in the art of pharmacy.

In managing the patient, the therapy may be initiated at a lower dose, e.g., from about 1 mg, and increased up to the recommended daily dose or higher depending on the patient's global response. It is further recommended that smaller animals, such as cats and those with impaired renal or hepatic function, initially receive low doses when administered systemically, and that they be titrated based on individual response(s) and blood level(s). It may be necessary to use dosages outside these ranges in some cases, as will be apparent to those of ordinary skill in the art. Furthermore, it is noted that the clinician or treating physician will know how and when to interrupt, adjust, or terminate therapy in conjunction with individual animal's response.

Any suitable route of administration may be employed for providing the animal with an effective dosage of metal oxide, or a pharmaceutically acceptable derivative thereof. The most suitable route in any given case will depend on the nature and severity of the condition being prevented, treated, or managed. One preferred route is parenterally, preferably intravenously. In this embodiment, a preferred intravenous route of administration is by infusion.

In practical use, metal oxide, or a derivative thereof, can be combined as the active ingredient in intimate admixture with a pharmaceutical carrier according to conventional pharmaceutical compounding techniques. The carrier may take a wide variety of forms and may include a number of components depending on the form of preparation desired for administration. The compositions of the present invention include, but are not limited to, suspensions, solutions and elixirs; aerosols; or carriers, including, but not limited to, starches, sugars, microcrystalline cellulose, diluents, granulating agents, lubricants, binders, disintegrating agents, and the like.

Another suitable route of administration of the silver tetroxide compositions of the invention is topically, e.g., either directly as a powder or in non-sprayable or sprayable form. Topical administration is a preferred route of administration for treating topical conditions. In one embodiment, the metal oxide may be applied topically to the affected skin areas directly in powder form or in compounded formulations.

Non-sprayable forms can be semi-solid or solid forms including a carrier indigenous to topical application and preferably having a dynamic viscosity greater than that of water. Suitable formulations include, but are not limited to, suspensions, emulsions, creams, ointments, powders, liniments, salves and the like. If desired, these may be sterilized or mixed with any available auxiliary agents, carriers, or excipients, e.g., thixotropes, stabilizers, wetting agents, and the like. One or more thixotropic agents can be included in types and amounts sufficient to increase adhesion of topically applied compositions of the invention to a surface or mucosa associated with a treatment zone such as, for example, the skin, vagina, or cervix, so as to inhibit or prevent runoff or other loss of the composition from the treatment zone, particularly when the compositions are formulated for topical administration. With respect to conditions associated with the skin, the compositions preferably prevent, treat, or manage such conditions or diseases without visibly staining the skin, i.e., no staining to the naked eye.

Preferred vehicles for non-sprayable topical preparations include ointment bases, e.g., polyethylene glycol-1000 (PEG-1000); conventional ophthalmic vehicles; creams; and gels, as well as petroleum jelly and the like. In one preferred topical embodiment, the carrier includes a petroleum jelly. In another preferred topical embodiment, the carrier is formulated as a cream, gel, or lotion. A preferred composition comprises about 3% metal oxide, such as tetrasilver tetroxide, about 47% white petrolatum, about 36% heavy mineral oil, and about 14% TIVAWAX P Tivian Laboratories Inc., Providence, R.I. These topical preparations may also contain emollients, perfumes and/or pigments to enhance their acceptability for various uses.

In a preferred embodiment, a metal oxide, or a derivative thereof, is formulated for parenteral administration by injection (subcutaneous, bolus injection, intramuscular, or intravenous, such as by infusion), and may be dispensed in a unit dosage form, such as a multidose container or an ampule. Parenteral administration is a preferred administration route when the cancer is systemic, i.e., has a locus inside the body. Preferably, the formulation adapted for parenteral administration includes an insufficient amount of persulfate to induce irritation or adverse side effects. In one preferred embodiment, the formulation is substantially free of added persulfate, while in another more preferred embodiment, the formulation is completely free of added persulfate.

When administered intravenously, such as by infusion, the dosage preferably provides a concentration of the metal oxide in the blood stream of about 1 ppm to about 75 ppm, more preferably from about 5 ppm to about 50 ppm, such as from about 10 ppm to about 40 ppm or about 50 to 200 mg. In a preferred embodiment, a one-time dosage is infused or injected directly into the bloodstream.

The intravenous dosage is preferably delivered over a period of time sufficient to substantially inhibit or even avoid the occurrence of side effects. For example, the dosage can be delivered by intravenously or by infusion over a time from about 10 minutes to about 300 minutes, preferably from about 20 minutes to about 240 minutes.

Compositions of the metal oxide, or a pharmaceutically acceptable derivative thereof, for parenteral administration may be in the form of suspensions, solutions, emulsions, or the like, in aqueous or oily vehicles, and in addition to the active ingredient may contain one or more formulary agents, such as dispersing agents, suspending agents, stabilizing agents, preservatives, and the like.

Pharmaceutical compositions of the present invention may be orally administered in discrete pharmaceutical unit dosage forms, such as capsules, cachets, soft elastic gelatin capsules, tablets, or aerosols sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the pharmaceutically acceptable carrier which constitutes one or more necessary ingredients. In general, the compositions are prepared by uniformly and intimately admixing the active ingredient with liquid carriers or finely divided solid carriers or both, and then, if necessary, shaping the product into the desired presentation. Suitable types of oral administration include oral solid preparations, such as capsules or tablets, or oral liquid preparations. If desired, tablets may be coated by standard aqueous or non-aqueous techniques.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, granulating agent, surface active agent, dispersing agent, or the like. Molded tablets may be made by molding, in a suitable machine, a mixture of the powdered compound moistened with an inert liquid diluent. In one embodiment, each tablet, capsule, cachet, or gel cap contains from about 0.5 mg to about 500 mg of the active ingredient, while in another embodiment, each tablet contains from about 1 mg to about 250 mg of the active ingredient. The amount of active ingredient found in the composition, however, may vary depending on the amount of active ingredient to be administered to the animal. The tablets can be added to the feed of the animals being treated.

Another suitable route of administration is transdermal delivery, for example, via an abdominal skin patch. Preferably, the region receiving the patch is shaved prior to application to ensure better transmission of the active ingredient.

The metal oxide, or a suitable derivative thereof, may be formulated as a pharmaceutical composition in a soft elastic gelatin capsule unit dosage form by using conventional methods well known in the art, such as in Ebert, Pharm. Tech, 1(5):44-50 (1977). Soft elastic gelatin capsules have a soft, globular gelatin shell somewhat thicker than that of hard gelatin capsules, wherein a gelatin is plasticized by the addition of plasticizing agent, e.g., glycerin, sorbitol, or a similar polyol. The hardness of the capsule shell may be changed by varying the type of gelatin used and the amounts of plasticizer and water. The soft gelatin shells may contain a preservative, such as methyl- and propylparabens and sorbic acid, to prevent the growth of fungi. The active ingredient may be dissolved or suspended in a liquid vehicle or carrier, such as vegetable or mineral oils, triglycerides, surfactants such as polysorbates, or a combination thereof.

The metal oxide can be delivered via a controlled release delivery vehicle. In a preferred embodiment, the controlled release vehicle includes a polymeric material, delivered or surgically implanted at or near a lesion site. One of ordinary skill in the art will be familiar with controlled release means and delivery devices, such as those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which are hereby incorporated herein by express reference thereto. These pharmaceutical compositions can be used to provide slow or controlled-release of the active ingredient therein using, for example, hydropropylmethyl cellulose in varying proportions to provide the desired release profile, other polymer matrices, gels, permeable membranes, osmotic systems, multilayer coatings, microparticles, liposomes, microspheres, or the like, or a combination thereof. Suitable controlled-release formulations available to those of ordinary skill in the art, including those described herein, may be readily selected for use with the the metal oxide compositions of the invention. Thus, single unit dosage forms suitable for topical, parenteral, or oral administration, such as infusions, intravenous drips, gels, lotions, cremes, tablets, capsules, gelcaps, caplets, and the like, that are adapted for controlled-release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations may include: 1) extended activity of the drug; 2) reduced dosage frequency; and 3) increased patient compliance.

Most controlled-release formulations are designed to initially release an amount of drug that promptly produces the desired therapeutic effect, and gradual and continual release of other amounts of drug to maintain this level of therapeutic effect over an extended period of time. In order to maintain this constant level of drug in the body, the drug should be released from the dosage form at a rate that will replace the amount of drug being metabolized and excreted from the body.

The controlled-release of the active ingredient may be stimulated by various inducers, for example pH, temperature, enzymes, water, or other physiological conditions or compounds. The pharmaceutical compositions for use in the present invention include the metal oxide, or a derivative thereof, as the active ingredient, and may also contain a pharmaceutically acceptable carrier, and optionally, other therapeutic ingredients. Suitable derivatives include any available “pharmaceutically acceptable salts,” which refer to a salt prepared from pharmaceutically acceptable non-toxic acids including inorganic acids, organic acids, solvates, hydrates, or clathrates thereof. Preferably, in the case of silver (I,III), the salts do not comprise halides. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, citric, fumaric, gluconic, isethionic, lactic, malic, mucic, tartaric, para-toluenesulfonic, glycolic, glucuronic, maleic, furoic, glutamic, salicylic, mandelic, methanesulfonic, ethanesulfonic, benzenesulfonic (besylate), sulfanilic, alginic, galacturonic, and the like. Particularly preferred acids phosphoric, methanesulfonic, and glycolic.

Although preferred embodiments of the invention have been illustrated in the foregoing description, it will be understood that the invention is not limited to the embodiments disclosed, but is capable of numerous rearrangements and modifications of parts and elements without departing from the spirit of the invention.

Another embodiment of the present invention relates to the use of oxidative fluorinator compounds to treat microbial infections in animals. The term “microbe” as used herein refers to bacteria, viruses, yeasts, and fungi. The term “antimicrobial” as used herein is intended to define a substance that kills or reduces microbes and/or prevents or inhibits the proliferation of microbes, and is intended to include, among other things, substances that are bactericidal, bacteriostatic, virucidal, virustatic, fungicidal and/or fungustatic.

The invention provides a method for treating conditions of animals that are associated with infection by one or more microbes. The microbes suitable for treatment include any of the above-mentioned microbes. Conditions suitable for treatment with compounds of the invention include, for example, any of the above mentioned conditions and in particular those conditions affecting mammals, such as cattle and domesticated animals. Particular diseases include, for example, Actinobacillosis, Anaplasmosis, Bovine babesiosis, Bovine ephemeral fever (BEF), Bovine brucellosis, Boophilus microplus, Haemorrhagic septicaemia (HS), Contagious bovine pleuropneumonia (CBPP), Rinderpest, Bovine tuberculosis (bovine TB), calf diphtheria, foot-and-mouth disease, bovine respiratory disease, feline immunodeficiency virus, and feline leukemia.

Methods of treatment include contacting the microbes with a composition including an effective amount of at least one oxidative fluorinator compound, the oxidative fluorinator compound including a fluoride that, when dissolved in aqueous solution, will provide at least one fluoride group that does not dissociate into a fluoride anion.

The invention provides a composition that inhibits or destroys microbes after contact of the microbe with the composition. Preferred formulations for the compositions of the invention include antibiotic compositions, virucidal compositions, fungicidal compositions, and bactericidal compositions.

The term “oxidative fluorinator” is a term recognized in the art and used by Bartlett and Skladky in Chemical Communications (1968), p. 1046, to describe fluorides that are capable of directly or indirectly generating species, e.g., elemental fluorine, which is the most electronegative and reactive element known. Thus, oxidative fluorinator compounds generate species that will react with other compounds by addition or displacement reactions, such as the replacement of hydrogen or other groups to form fluorinated compounds, particularly fluorinated hydrocarbons.

Many water-soluble fluoride salts in aqueous solution dissociate exclusively into cations and fluoride anions F⁻. For example, NaF dissociates to Na⁺ and F⁻, CoF₂ dissociates into Co²⁺ and 2 F⁻. These and other fluorides cannot be characterized as fluorinator compounds.

The oxidative fluorinator compounds of the invention, however, do not dissociate in that way. For example, cobalt trifluoride, unlike its divalent counterpart, will not yield three fluoride ions upon dissociation. Actually, CoF₃ is a source of elemental fluorine and as such, the dissociation product of CoF₃ provides at least one fluoride group that does not dissociate to a fluoride anion, and also provides, either directly or indirectly, elemental fluorine or a source of elemental fluorine. Cobalt trifluoride is described as a fluorinator in chemical synthesis. See, e.g., M. Stacey et al., Adv. Fluorine Chem. 1,166 (1960); A. Me Killop et al., J. Am. Chem. Soc. 102, 6504 (1980). Other fluorides described in the literature as fluorinator compounds include xenon fluorides, manganese tetrafluoride, and nickel tetrafluoride.

The present invention is based on the discovery that oxidative fluorinator compounds, such as described above, are effective antimicrobial agents, even when used at very low levels of less than about 20 ppm by weight in antimicrobial compositions, or when added to swimming pools or industrial cooling water at less than about 20 ppm by weight, to control microbes, such as bacteria.

Suitable oxidative fluorinator compounds of the invention include, but are not limited to, tri- or tetravalent transition metal fluorides, inert gas fluorides, such as xenon fluorides or krypton fluorides, tri- or tetravalent rare earth metal fluorides, oxyfluorides such as those of vanadium and hydrogen, or mixtures thereof.

Suitable tri- or tetravalent transition metal fluorides include cobalt trifluoride, nickel tetrafluoride, manganese tetrafluoride, and combinations thereof.

Preferred fluorides include higher atomic weight inert gas fluorides, such as krypton fluorides and xenon difluoride, xenon tetrafluoride, and xenon hexafluoride. Rare earth metal fluorides are also suitable, particularly those of the cerium subgroup such as praseodymium tetrafluoride.

In addition, crystalline adducts of xenon fluorides and metal oxidative fluorinator compounds are also preferred fluorinator compounds, such as the adduct of XeF₆ and MnF₄ (4XeF₆. MnF₄) and the adduct of XeF₆ and PrF₄ (XeF₆.4PrF₄). Preferred oxidative fluorinator compounds forming such adducts are transition or rare earth metal tetrafluorides.

All of these represent fluorides that do not completely dissociate into ions in water and will provide reactive fluorination species having antimicrobial efficacy according to the invention when dissociated.

More preferred compositions, particularly those based on transition metal fluorides, also contain a strong oxidizer that functions to stabilize the oxidation states of the fluorides when dissolved in aqueous solutions. In particular, the transition metal fluorides can be used in combination with a strong oxidizer, preferably having a reduction potential EMF of greater than about 2 volts. Suitable oxidizers include potassium and sodium persulfates, more preferably potassium monopersulfate. The strong oxidizer is believed to stabilize the oxidation state of the more unstable metal fluorides in an aqueous medium.

Tests have shown that inert gas fluorides, such as xenon fluorides, are more stable than the transition metal fluorides and the strong oxidizer is optional for these materials to maintain their efficacy as antimicrobials. These materials, however, can also be effectively stabilized using an inorganic acid, such as nitric acid, or more preferably phosphoric acid.

The most preferred oxidative fluorinator compounds of this invention are xenon difluoride, with or without a strong oxidizer, and cobalt trifluoride when used in combination with a strong oxidizer.

The oxidative fluorinator compounds of the invention exhibit a surprising efficacy in killing, or inhibiting, or preventing the proliferation of, large colonies of various bacteria, viruses, fungi, and other microbes after only a short period of contact. The oxidative fluorinator compounds have been found to provide high, or even complete, kill rates for microbes when the microbes are contacted with solutions containing less than about 20 ppm by weight of the fluorinator, and even at concentrations of less than about 10 ppm, or from about 0.1 ppm to about 5 ppm. The exact mechanism is not known, but is believed to be related to redox or microbe fluorination. It should be understood that amounts greater than about 20 ppm of oxidative fluorinator are also included in the invention, for example, about 20 ppm to about 20,000 ppm, about 50 ppm to about 5,000 ppm, and about 100 ppm to about 1,000 ppm, in varying embodiments.

Where the oxidative fluorinator compositions according to the invention are applied to the skin to treat conditions associated with microbial infection, they may be combined with a carrier at an amount from about 5 ppm to about 500,000 ppm, more preferably from about 50 ppm to about 250,000 ppm of the oxidative fluorinator composition, based on the weight of the carrier. In various embodiments, the compositions are provided in amounts from about 400 ppm to about 100,000 ppm, from about 1,000 ppm to about 70,000 ppm, from about 10,000 ppm to about 50,000 ppm, or from about 20,000 ppm to about 40,000 ppm. In one preferred embodiment, the compositions are formulated with about 25,000 ppm to about 35,000 ppm of oxidative fluorinator compound. It will be readily understood by those of ordinary skill in the art that 1 ppm of oxidative fluorinator compound is approximately equivalent to 1 mg/L. The compositions, when applied topically, can be applied to the skin about 1 to about 3 times per day until the condition is suitably cured or satisfactorily controlled. In one embodiment, the composition may generally be topically applied at a dosage level of from about 1 mg to about 1000 mg per cm² of skin surface, preferably about 10 mg to about 500 mg per cm² of skin surface. A preferred carrier includes petroleum jelly, such as white petroleum jelly.

In practical use, oxidative fluorinator, or a derivative thereof, can be combined as the active ingredient in intimate admixture with a pharmaceutical carrier according to conventional pharmaceutical compounding techniques. The carrier may take a wide variety of forms and may include a number of components depending on the form of preparation desired for administration. The compositions of the present invention may include, but are not limited to, suspensions, solutions and elixirs; aerosols; or carriers, including, but not limited to, starches, sugars, microcrystalline cellulose, diluents, granulating agents, lubricants, binders, disintegrating agents, and the like.

The fluorinator compounds may also be formulated into anti-bacterial cleaning compositions (liquids or powders) or laundry detergents. The fluorinator compounds may further be formulated into compositions containing one or more additional antimicrobial agents, such as antibiotics or other germicides.

A preferred route of administration of the oxidative fluorinator compounds of the invention is topically, e.g., either directly as a powder or in non-sprayable or sprayable form. Non-sprayable forms can be semi-solid or solid forms including a carrier indigenous to topical application and preferably having a dynamic viscosity greater than that of water. Suitable formulations include, but are not limited to, suspensions, emulsions, creams, ointments, powders, liniments, salves and the like. If desired, these may be sterilized or mixed with one or more of any available auxiliary agents, carriers, or excipients, e.g., thixotropes, stabilizers, wetting agents, and the like, and combinations thereof. One or more thixotropic agents can be included in types and amounts sufficient to increase adhesion of topically applied compositions of the invention to the skin, so as to inhibit or prevent runoff or other loss of the composition from the treatment zone on the skin. Preferred vehicles for non-sprayable topical preparations include ointment bases, e.g., polyethylene glycol-1000 (PEG-1000); conventional ophthalmic vehicles; creams; and gels, as well as petroleum jelly and the like. In one more preferred embodiment, the carrier includes a petroleum jelly. In another preferred embodiment, the carrier is formulated as a cream, gel, or lotion. In another preferred embodiment, the carrier is about 3 weight percent active ingredient, about 36 weight percent heavy mineral oil, about 47 weight percent petroleum jelly, and about 14 weight percent Tivawax P, available from Tivian Laboratories, Inc., of Providence, R.I. In yet another preferred embodiment, the carrier may be a dry powder compositions, such as with about 5 weight percent active ingredient and about 95 weight percent bismuth subgallate.

The pharmaceutical compositions for use in the present invention include oxidative fluorinator, or a derivative thereof, as the active ingredient, and may also contain a pharmaceutically acceptable carrier, and optionally, other therapeutic ingredients. Suitable derivatives include any available “pharmaceutically acceptable salts,” which refer to a salt prepared from pharmaceutically acceptable non-toxic acids including inorganic acids, organic acids, solvates, hydrates, or clathrates thereof. Examples of such inorganic acids are nitric, sulfuric, lactic, glycolic, salicylic, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Particularly preferred acids are lactic, glycolic, and salicylic acids.

In addition to the common dosage forms set out above, the compounds of the present invention may also be administered by controlled release means, delivery devices, or both, as are well known to those of ordinary skill in the art, such as those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which are hereby incorporated herein by express reference thereto. These pharmaceutical compositions can be used to provide slow or controlled-release of the active ingredient therein using, for example, hydropropylmethyl cellulose in varying proportions to provide the desired release profile, other polymer matrices, gels, permeable membranes, osmotic systems, multilayer coatings, microparticles, liposomes, microspheres, or the like, or a combination thereof. Suitable controlled-release formulations available to those of ordinary skill in the art, including those described herein, may be readily selected for use with the oxidative fluorinator compounds of the invention. Thus, single unit dosage forms suitable for topical administration, such as gels, lotions, cremes, and the like, that are adapted for controlled-release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations may include: 1) extended activity of the drug; 2) reduced dosage frequency; and 3) increased patient compliance.

Most controlled-release formulations are designed to initially release an amount of drug that promptly produces the desired therapeutic effect, and gradual and continual release of other amounts of drug to maintain this level of therapeutic effect over an extended period of time. In order to maintain this constant level of drug in the body, the drug should be released from the dosage form at a rate that will replace the amount of drug being metabolized and excreted from the body.

The controlled-release of the active ingredient may be stimulated by various inducers, for example pH, temperature, enzymes, water, or other physiological conditions or compounds. The term “controlled-release component” in the context of the present invention is defined herein as a compound or compounds, including polymers, polymer matrices, gels, permeable membranes, liposomes, microspheres, or the like, or a combination thereof, that facilitates the controlled-release of the active ingredient (e.g., oxidative fluorinator) in the pharmaceutical composition.

### EXAMPLES

These and other aspects of the present invention may be more fully understood with reference to the following examples, which are merely illustrative of the preferred embodiments of the present invention, and are not to be construed as limiting the invention, the scope of which is defined by the appended claims. Unless otherwise indicated, antimicrobial evaluations of the various fluorinator compounds tested were performed versus control samples in cultures in accordance with the protocol as set forth in AOAC (15th) 1990:965:13 at colony densities indicated in the examples.

### Example 1

Contaminated water was prepared and measured to have AOAC coliform counts averaging 37.4 colony forming units per milliliter (cfu/mL). The water was treated by first adding crystal potassium monopersulfate (KPS) stabilizer, commercially available from E.I. DuPont de Nemours & Co. Corp. as OXONE®, and xenon difluoride (XeF₂). The concentration in solution of the KPS and XeF₂ was about 20 PPM and about 2 PPM respectively, and the pH of the treated solution was about 7. Culture test versus controls showed that the coliform were inhibited by about 73% after 5 minutes of exposure time and by about 77% after 10 minutes of exposure time.

### Example 2

Aqueous solutions were prepared having a salmonella contamination at a cell density of about 500,000 cfu/mL. Two solutions of XeF₂ were prepared: one as a 1% solution in 85% phosphoric acid stabilizer, and a second solution containing no stabilizer. The materials were cultured versus controls at concentrations of about 2 ppm and about 4 ppm XeF₂ and at a pH of about 9. After ten minutes of exposure time, the phosphoric acid-stabilized XeF₂ culture exhibited an inhibition of about 93.2%, while the XeF₂ culture had an inhibition of only about 13.2%. At about 4 ppm XeF₂, however, the former exhibited an inhibition of 100% and the latter about 96.4%.

### Example 3

The inhibition % of CoF₃ stabilized with about 10 ppm KPS at a pH of about 7 was evaluated as in Example 2 against E. Coli at various cell densities. The results are shown in Table 1.

TABLE 1

| | Sample 1 | Sample 2 | Sample 3 |
|---|---|---|---|
| Cell Density (cfu/mL) | 200,000 | 420,000 | 600,000 |
| CoF₃ (ppm) | 2 | 1 | 0.5 |
| Contact Time (min.) | 0.5 | 10 | 10 |
| Inhibition (%) | 98 | 100 | 97.5 |

Thus, the data show that the oxidative fluorinator compounds of this invention are capable of killing microbes up to at least about 1,000,000 cfu/mL at concentrations of up to about 10 ppm. The data further indicate antimicrobial action against Gram Negative and Gram Positive microorganisms as well as yeast and mold.

It will be understood that the claims are intended to cover all changes and modifications of the preferred embodiments of the invention, herein chosen for the purpose of illustration, which do not constitute a departure from the spirit and scope of the invention.

What is claimed is:

1. A method for preventing, treating, or managing foot and mouth disease in an animal, which method comprises:
administering at least one metal oxide compound or a pharmaceutically acceptable derivative thereof, to the animal in an amount and for a period of time which is therapeutically effective, wherein each metal oxide compound or derivative thereof comprises a first metal cation having a first valence state and a second metal cation having a second, different valence state.

2. The method of claim 1, wherein the at least one metal oxide compound comprises at least one electron active metal oxide compound.

3. The method of claim 2, wherein the at least one metal oxide compound or pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

4. The method of claim 1, wherein the foot and mouth disease is associated with infection by Aphthovirus.

5. The method of claim 1, wherein the metal oxide compound or derivative thereof, is administered via intravenous injection or infusion.

6. The method of claim 4, wherein the administering is subcutaneous, intramuscular, or by infusion into a blood stream of the animal.

7. The method of claim 5, wherein the metal oxide compound or derivative thereof is administered in an amount sufficient to provide about 1 to about 75 ppm of the metal oxide compound or derivative thereof in the bloodstream.

8. The method of claim 6, wherein the metal oxide compound or derivative thereof is administered via infusion over a period of from about 30 minutes to about 300 minutes to inhibit adverse side effects.

9. The method of claim 8, wherein the at least one other chemotherapeutic agent is administered concurrently with the metal oxide compound or derivative thereof.

10. The method of claim 1, wherein the metal oxide compound or derivative thereof is administered by a controlled release vehicle.

11. The method of claim 10, wherein the controlled release vehicle is implanted in the body at a location suitable for providing a therapeutically effective amount of metal oxide compound or derivative thereof to the animal without affecting proper functioning of the animal's liver.

12. The method of claim 1, wherein the metal oxide compound or derivative thereof is substantially free of added persulfate.

13. A method for preventing, treating, or managing feline leukemia virus in a mammal, comprising administering at least one metal oxide compound or a pharmaceutically acceptable derivative thereof, to the animal in an amount and for a period of time which is therapeutically effective, wherein each metal oxide compound or derivative thereof comprises a first metal cation having a first valence state and a second metal cation having a second, different valence state.

14. The method of claim 13, wherein the at least one metal oxide compound or a pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

15. The method of claim 13, wherein the at least one metal oxide compound or a pharmaceutically acceptable derivative thereof is substantially free of added persulfate.

16. The method of claim 13, wherein the therapeutically effective amount is from about 10 ppm to 100,000 ppm.

17. A method for treating a bovine respiratory disease, comprising comprising administering at least one metal oxide compound or a pharmaceutically acceptable derivative thereof, to the animal in an amount and for a period of time which is therapeutically effective, wherein each metal oxide compound or derivative thereof comprises a first metal cation having a first valence state and a second metal cation having a second, different valence state.

18. The method of claim 17, wherein the at least one metal oxide compound or a pharmaceutically acceptable derivative thereof comprises at least one of Bi(III,V) oxide, Co(II,III) oxide, Cu(I,III) oxide, Fe(II,III) oxide, Mn(II,II) oxide, Pr(III,IV) oxide, or Ag(I,III) oxide.

19. The method of claim 17, wherein the respiratory disease is associated with infection by at least one of P. haemolytica, P. multocida, bovine herpes virus 1, parainfluenza-3, bovine respiratory syncytial virus, BRSV, Haemophilus somnus, Pasteurella spp., P13, IBR, BVDV and Mycoplasma.

20. A method for treating a feline immunodeficiency disease, comprising administering at least one metal oxide compound or a pharmaceutically acceptable derivative thereof, to the animal in an amount and for a period of time which is therapeutically effective, wherein each metal oxide compound or derivative thereof comprises a first metal cation having a first valence state and a second metal cation having a second, different valence state.

21. The method of claim 20, wherein the at least one metal oxide compound or a pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

22. The method of claim 2, wherein the at least one metal oxide compound or a pharmaceutically acceptable derivative thereof is substantially free of added persulfate.

23. The method of claim 20, wherein the therapeutically effective amount is from about 10 ppm to 100,000 ppm.

24. A method for treating a disease of an animal, the method comprising administering at least one metal oxide compound or a pharmaceutically acceptable derivative thereof, to the animal in an amount and for a period of time which is therapeutically effective, wherein each metal oxide compound or derivative thereof comprises a first metal cation having a first valence state and a second metal cation having a second, different valence state, wherein the disease comprises at least one of Actinobacillosis, Anaplasmosis, Bovine babesiosis, Bovine ephemeral fever (BEF), Bovine brucellosis, Boophilus microplus, Haemorrhagic septicaemia (HS), Contagious bovine pleuropneumonia (CBPP), Rinderpest, Bovine tuberculosis (bovine TB), calf diphtheria, foot-and-mouth disease, bovine respiratory disease, feline immunodeficiency virus, and feline leukemia.

25. The method of claim 24, wherein the metal oxide is essentially free of tetrasilver tetroxide.

26. The method of claim 24, wherein the at least one metal oxide compound or a pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

27. The method of claim 24, wherein the at least one metal oxide compound or a pharmaceutically acceptable derivative thereof is substantially free of added persulfate.

28. The method of claim 24, wherein the therapeutically effective amount is from about 10 ppm to 100,000 ppm.

29. A method of preventing, treating, or managing a condition of a mammal, which method comprises administering a therapeutically effective amount of at least one electron active compound, or a pharmaceutically acceptable derivative thereof, that has at least two polyvalent cations, at least one of which has a first valence state and at least one of which has a second different valence state, to prevent, treat, or manage the condition, or a symptom thereof.

30. The method of claim 29, wherein the electron active compound does not include tetrasilver tetroxide.

31. The method of claim 29, wherein the mammal is a cow, a pig, a sheep, a goat, a deer, or a horse.

32. The method of claim 29, wherein the condition is associated with infection by at least one of a bacteria and a virus.

33. The method of claim 29, wherein the at least one metal oxide compound or a pharmaceutically acceptable derivative thereof comprises at least one of Bi(III,V) oxide, Co(II,III) oxide, Cu(I,III) oxide, Fe(II,III) oxide, Mn(II,III) oxide, Pr(III,IV) oxide, or Ag(I,III) oxide.

34. The method of claim 33, wherein the at least one metal oxide compound or a pharmaceutically acceptable derivative thereof does not include tetrasilver tetroxide.

35. The method of claim 29, wherein the at least one metal oxide compound or a pharmaceutically acceptable derivative thereof is substantially free of added persulfate.

36. The method of claim 29, wherein the therapeutically effective amount is from about 10 ppm to 100,000 ppm.

37. A method for destroying or inhibiting proliferation of microbes associated with an infection of an animal, which method comprises contacting the microbes with a composition comprising an amount of at least one oxidative fluorinator compound that provides at least one fluoride group that does not dissociate into a fluoride anion when dissolved in an aqueous solution, wherein the amount of the compound is effective to destroy the microbes or inhibit proliferation.

38. The method of claim 37, wherein the composition further comprises at least one oxidizing agent.

39. The method of claim 38, wherein the at least one oxidizing agent comprises at least one of sodium persulfate or potassium persulfate.

40. The method of claim 38, wherein the at least one oxidizing agent comprises potassium monopersulfate.

41. The method of claim 37, wherein the composition comprises a combination of at least two different fluorinator compounds.

42. The method of claim 37, wherein the composition comprises about 0.1 to about 10 ppm by weight of the fluorinator compound.

43. The method of claim 39, wherein the composition comprises about 0.1 to about 50 ppm by weight sodium persulfate or potassium persulfate and about 0.1 to about 10 ppm by weight fluorinator compound.

44. The method of claim 37, wherein the fluorinator compound comprises at least one of tri- or tetravalent transition metal fluorides, inert gas fluorides, tri- or tetravalent rare earth metal fluorides, oxyfluorides, or mixtures thereof.

45. The method of claim 37, wherein the fluorinator compound comprises at least one of cobalt trifluoride, nickel tetrafluoride, manganese tetrafluoride, xenon difluoride, xenon tetrafluoride, xenon hexafluoride, or mixtures thereof.

46. The method of claim 37, wherein the fluorinator compound comprises an adduct of xenon fluoride, or a transition metal oxidative fluorinator compound.

47. The method of claim 37, wherein the fluorinator compound comprises an adduct of xenon fluoride and a rare earth metal oxidative fluorinator compound.

48. The method of claim 37, wherein the composition further comprises an acidic stabilizer.

49. The method of claim 48, wherein the acidic stabilizer comprises phosphoric acid.

50. The method of claim 37, wherein the fluorinator compound completely prevents microbe proliferation.

51. The method of claim 37, wherein the microbes comprise at least one of a bacteria, virus, or fungus.

52. A method for destroying or inhibiting proliferation of microbes associated with an infection of an animal, which method comprises contacting the microbes with a composition comprising a mixture of about 0.1 to about 50 ppm by weight of an alkali metal persulfate and about 0.1 to about 20 ppm by weight of cobalt trifluoride.

53. A composition for destroying or inhibiting proliferation of microbes associated with an infection of an animal which comprises at least one oxidative fluorinator compound that provides at least one fluoride group that does not dissociate into a fluoride anion when dissolved in an aqueous solution, wherein the compound is present in an amount effective to destroy microbes or inhibit proliferation.

54. The composition of claim 53 formulated as a lotion, balm, aerosol spray, ointment, gel, or shampoo.

55. The composition of claim 53, further comprising at least one oxidizing agent.

56. The composition of claim 55, comprising about 0.1 to about 50 ppm by weight of an alkali metal persulfate and about 0.1 to about 20 ppm by weight fluorinator compound.

57. The method of either claim 52 or claim 53, wherein the infection comprises at least one of Actinobacillosis, Anaplasmosis, Bovine babesiosis, Bovine ephemeral fever (BEF), Bovine brucellosis, Boophilus microplus, Haemorrhagic septicaemia (HS), Contagious bovine pleuropneumonia (CBPP), Rinderpest, Bovine tuberculosis (bovine TB), calf diphtheria, foot-and-mouth disease, bovine respiratory disease, feline immunodeficiency virus, and feline leukemia.
